(19) United States
(12) Patent Application Publication
(10) Pub. No.: US 2019/0240458 A1
(43) Pub. Date: Aug. 8, 2019

(54) A MEDICAL TUBE STORAGE SYSTEM

(71) Applicant: TOOKIE LIMITED, Liverpool (GB)

(72) Inventor: Stephen TOOKE, Liverpool (GB)

(21) Appl. No.: 16/339,875

(22) PCT Filed: Oct. 6, 2017
(86) PCT No.: PCT/GB2017/053028

§ 371 Date: Apr. 5, 2019

(30) Foreign Application Priority Data
Oct. 6, 2016 (GB) 1617001.1

(51) Int. Cl. A61M 25/02; A41D 13/12; A41D 27/20; A41D 1/04

(52) U.S. Cl. CPC A61M 25/02; A41D 13/1245; A61M 25/0017; A41D 1/04; A41D 27/20

(57) ABSTRACT

A medical tube holder comprising a vest having at least one sealable pocket located on the vest. The inside of the pocket is provided with at least one tube retaining devices into which the end of a medical tube may be releasably retained.

### FIELD OF THE INVENTION

The invention relates to a medical tube holder, particularly for use in patients with a long-term tube implanted.

### BACKGROUND TO THE INVENTION

It is well-known to implant medical tubes into patients and to have one end of the tube protruding from the patient. For example, central venous access catheters (“central lines”), and in particular so-called “Hickman lines”, are often required to be fitted in patients for extended periods. These lines are frequently used to administer medication or fluids and to obtain blood from a patient for testing. The central lines may be kept in place for several months at a time.

There are various complications with such central lines, including bloodstream infection due to bacteria ingress through the central line, which can be particularly damaging in cancer patients that may have become immunocompromised as a result of chemotherapy.

The risk of bacterial infection is particularly prevalent those that are less inclined to take care to keep the central line clean. Additionally, some patients may be more prone to mechanical damage to the central line by, for example, catching it on objects or pulling at it.

The same applies to animals that may have medical tubes implanted long-term that protrude from the patient.

Whilst previously it has been known to affix the end of the tube to the patient's skin, for example with medical tape, the end of the tube is still exposed to bacteria and the tape can become irritating, leading the patient to scratch at it, thereby increasing the risk of mechanical damage to the tube and the patient.

In patients suffering from renal complications, it is often necessary to employ a venous catheter than is inserted into the neck or chest. A consequence of this is that the catheter is located high up on the patient and should not be pulled. Therefore, where some tube holders position a tube end receiving portion at waist height, this may not be appropriate for renal patients.

### SUMMARY OF THE INVENTION

Accordingly, the present invention is directed to a medical tube holder comprising a vest and at least one sealable pocket located on the vest, wherein the inside of the pocket is provided with at least one tube retaining device into which the end of a medical tube may be releasably retained.

By providing a vest with a pocket, a patient is provided with a convenient storage location for the end of a medical tube, for example a central line, which can be worn. Such a vest thus provides a safe and secure manner in which to store the end of a protruding medical tube that moves with the patient. Additionally, the pocket may be lined to further protect the end of the tube, particularly if the lining is anti-bacterial and/or anti-microbial. The pocket may be provided on the internal and/or external surface of the vest. Where the pocket is on the external surface of the device, an aperture may be provided through which the medical tube can pass from the inside of the device to the outside. The diameter of that aperture may be variable by way of an adjustable and/or releasable fastening, such as hook-and-eye components.

The position of the pocket may depend upon the position of the exit site of the line from the patient, the type of line and reason for the line to be inserted. For example, the pocket may be positioned on the rear of the vest where the line protrudes from a user's neck or on the front of the vest where the line protrudes from the arm, chest or torso. The pocket may be aligned substantially laterally across the vest in order to retain the end of the tube in a substantially linear manner across the torso of the patient. This reduces the risk of fatigue and/or damage to the tube that might be caused by bending the tube to fit it into a pouch or pocket.

Preferably, the tube retaining device is a clip into which a medical tube can be received. It is envisaged that the tube retaining device may be a snap-fit clip that is resiliently yieldable to allow the tube to be positioned therein. Alternatively, or additionally, other types of tube retaining devices may be used for retaining the tubes, for example, different types of clips and/or soft retainers. “Soft retainers” are those that are able to be flattened without providing significant resistance, for example, releasable loops of material, which may be provided with opening means, such as hook-and-eye connectors, that will yield under a force thereagainst. Alternative retaining means and/or devices may include, but are not limited to, portions with apertures therethrough, loops, releasable loops, hooks and rings that may comprise textiles, fabrics and/or plastics materials. Where the part is releasable, the connection mechanism may be one or more of those mentioned herein, for example, hook-and-eye or push-fit connectors. Preferably, the retaining device is readily releasable to allow quick release of the tube. The retaining device may be in the form of a series, or plurality, of loops within the pocket for retaining of the tube along the length of the pocket. The series of loops may be in the form of a plurality of loops aligned longitudinally along the pocket. The use of a plurality of loops allows the device to compensate for the position of the tube on the patient and differing lengths of tubes between patients. Additionally, should the tube be shortened, the device is still able to function by employing a different loop in the series.

It is advantageous that the vest comprises at least one connection system to allow the vest to be opened. This may be in the form of at least one sealable opening to allow repeated removal of the device, or it may be in the form of a system that permanently breaks the vest.

The vest may be substantially one-use and disposable, in which case, use of the latter option may allow quick removal of the vest in an emergency. However, it may be advantageous that the resealable opening is employed to allow the vest to be repeatedly worn and removed from about the torso. Where a sealable opening is used, it may comprise one or more of: a zipper; hook-and-eye fastener; and snap-fit connectors.

The vest may include at least one shoulder strap that is adjustable in length. The shoulder strap(s) may be releasably connected to the front panel and/or back panel of the vest, which allows the vest to be readily put on and taken off. This releasable connection may be in the form of hook-and-eye fastener, clips, snap-fit connectors or other connection means. The length of the strap(s) may be adjusted by changing to position to which the strap(s) connect to the front panel of the vest or by providing length adjustment along the strap(s).

In one embodiment, the vest comprises a front panel and a back panel with two shoulder straps connecting an upper part of the front panel to an upper part of the back panel and a head aperture between the shoulder straps. The panels may be joined at the sides and the panels may be integral with one another. Where the vest is provided with a sealable opening, the opening may be positioned in the front or back panels or at the side of the vest. Alternatively, the straps may release to allow the vest to be readily removed as one would a jumper.

In a preferred arrangement, the vest comprises at least one aperture through which a medical tube may pass and, more preferably, the area of the at least one aperture is adjustable, this is to say, the area enclosed by the perimeter of the aperture may be increased or decreased. The aperture is, preferable, not within the pocket and, more preferably, the aperture is provided in a front panel of the device with the pocket being attached to the same, advantageously the pocket extending laterally across the front panel. The aperture may be part of the means for fastening the vest, but it is preferably a separate element in a separate position The use of an aperture through which a medical tube can pass provides a clear path for the tube to pass from the patient through to the front of the vest. Additionally, the vest can provide protection to the exit site, which may be near the user's clavicle and so the aperture may be in a corresponding position on the device. Where the area of the at least one aperture is adjustable, which may be put into effect by the position at which the straps connect to the front panel, the aperture and vest can be adjusted to retain the tube in the most appropriate manner. Additionally, or alternatively, the height of the aperture relative to the pocket of the vest and/or user's torso may be adjusted according to the position of the exit site.

The vest may comprise a plurality of pockets in different positions. There may be a plurality of pockets arranged on the front, back and/or sides of the vest according to demand Additionally, or alternatively, a pocket may be positioned on the straps, where present. Furthermore, it may be possible to attach further pockets to the vest, as required.

In one embodiment, the pocket comprises;
a first surface; and
a second surface designed to at least partially overlay the first surface;

wherein the first surface and the second surface are pivotally connected along at least a first edge, and

wherein at least a second edge of the first surface and/or second surface is provided with connecting means for releasably connecting the surfaces together.

The first and second surfaces of the pocket may be substantially rectangular and an edge of the first and/or second surface distal from the pivot is provided with attachment means. Having a single edge of connecting means, regardless of the shape of the pocket, allows for some lateral movement of the tube within the pocket, which can compensate for flexing of the device due to natural movements of the user, thereby reducing the risk of mechanical damage to the tube or exit site. Additionally, such an arrangement retains the tube with a closed pocket but allows quick and easy access in an emergency. Therefore, whilst it is envisaged that the connecting means may cover the majority or all of the periphery, providing a single edge with connecting means may, in some circumstances, be preferred. For the sake of completeness, two sides may be connected and one or more sides left clear, which will vary according to the shape of the holder.

Advantageously, padding is provided adjacent the pocket. The front and/or back panel may comprise an inner padding layer that is positioned adjacent the user, or they may comprise two outer layers with padding therebetween. This cushions the user against impact and, where hard tube retaining clips are employed, it absorbs any pressure thereupon.

The vest may comprise laterally extending straps connecting front and back panels to keeps the device held on the patient and reduces the likelihood of it moving relative to the patient's torso. This is particularly useful when the patient is sleeping. The laterally extending straps in combination with the shoulder straps also reduce longitudinal movement of the tube relative to the patient. Additionally, the laterally extending straps provide a convenient location to which items may be attached.

The vest may be back, front or side fastening, according to the needs of the user. For example, in children, it may be an advantage for the vest to be back-fastening to make it more difficult for the child to remove it. However, in adults, it may be preferred to have front or side fastening to allow more independence.

The pocket may comprise a liner. The use of a liner, and in particularly a removable liner that is anti-bacterial and anti-microbial, allows the end of the tube to be wrapped in the liner to reduce the risk of it being exposed to potentially harmful bacteria.

It is preferred that the vest comprises at least one anti-bacterial and/or anti-microbial material selected from a group comprising: polyester warp spacer fabric; paper; and bamboo yarn. The device may be manufactured from materials that are naturally anti-bacterial and/or antimicrobial, or they materials may be treated so that they are.

The vest may be seamless in order to increase comfort.

The invention extends to a method of using the device herein described.

### BRIEF DESCRIPTION OF THE DRAWINGS

An embodiment of the invention will now be described, by way of example only, and with reference to the accompanying drawings, in which:

FIG. 1 is a drawing showing a front view of a first device in accordance with the present invention;

FIG. 2 is a further drawing of the device of FIG. 1;

FIG. 3 is a drawing of a rear view of the device of FIG. 1;

FIG. 4 is a drawing of a tube retaining arrangement for use with the present invention;

FIG. 5 is drawing of a front view of a second device in accordance with the present invention;

FIG. 6 is a rear view of the device of FIG. 5;

FIG. 7 is a front view of a further embodiment of a device in accordance with the present invention;

FIG. 8 shows a perspective view of a second device in accordance with the present invention; and

FIG. 9 shows a view of the device of FIG. 8.

### DETAILED DESCRIPTION OF EXEMPLARY EMBODIMENTS

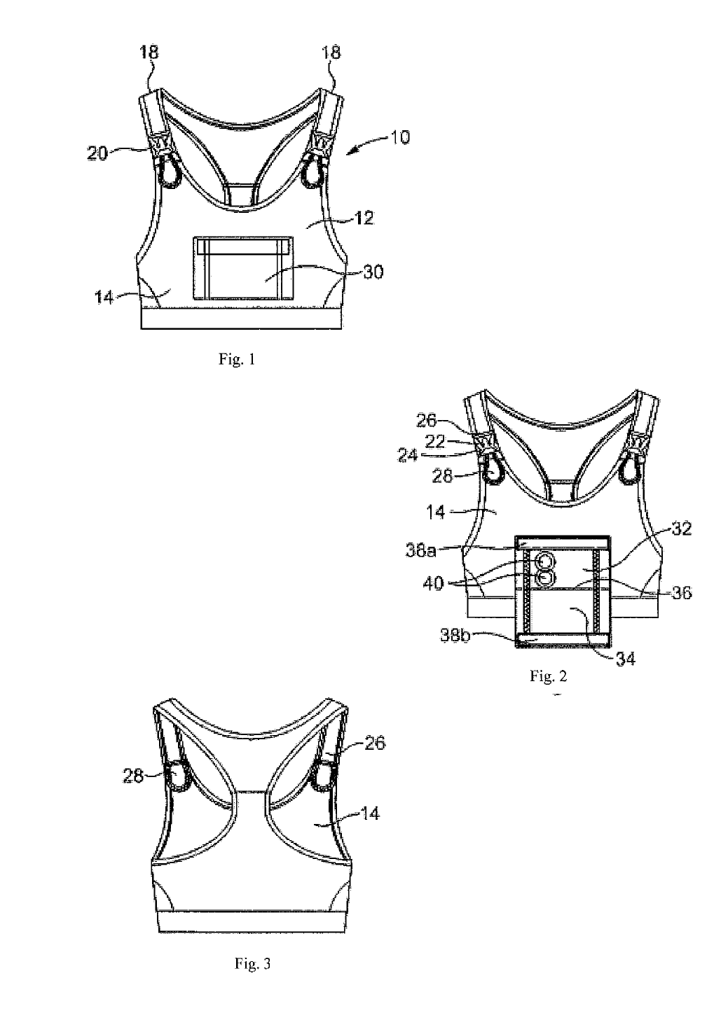

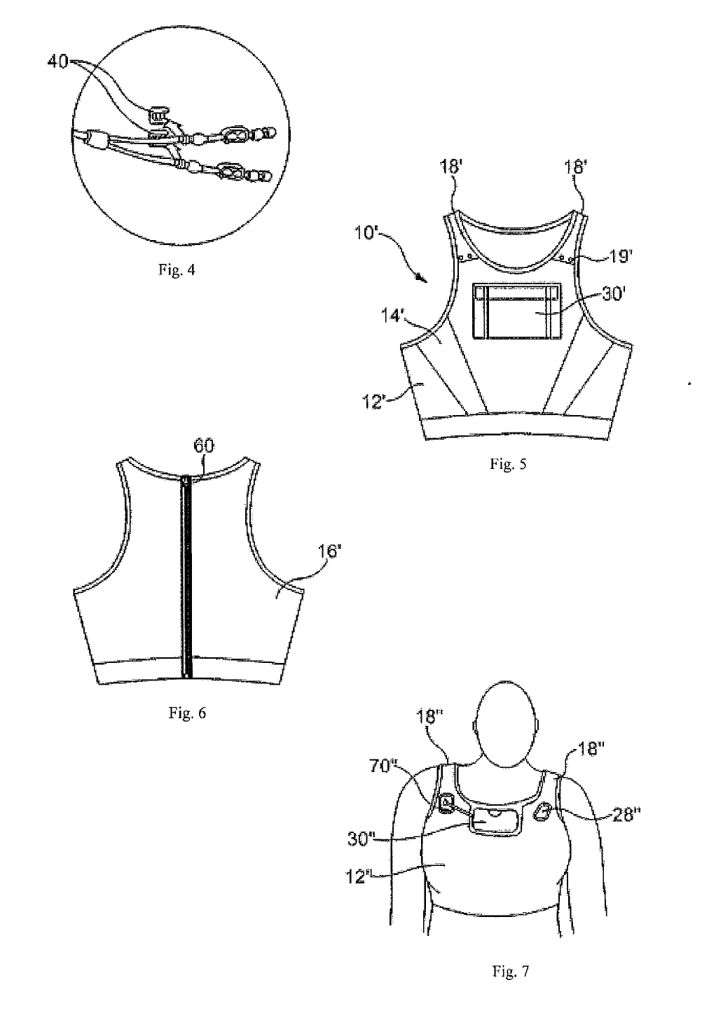

FIGS. 1 to 3 show a medical tube holder 10 in the form of a vest 12. The vest 12 comprises a front section 14 and a rear section 16 that are connected at their respective side edges. The top of the rear panel 16 splits into two shoulder straps 18 that connect to the top of the front panel 14 via a connection mechanism 20. The connection mechanism 20 comprises respective clips 22 on each shoulder strap 18 that are releasably received within respective receiving parts 24 on the front panel 14. A layer of material 26 is arranged behind the clips 22 to protect a user's skin from being caught within the clip 22 when received within the receiving part 24. An aperture 28 is positioned below the connection mechanism 20, which is sized and shaped to allow the passage of a medical tube therethrough. The vest 12 is seamless and is put on and/or removed over a user's head.

The straps 18 may be provided with a mechanism (not shown) to adjust their length to adjust the position of the apertures 28 relative to the user's torso.

The front panel 14 is provided with a pocket 30 comprising a first section 32 that is integrally, pivotally connected to a second section 34 along a fold line 36 such that the first section 32 and the second section 34 can at least partially overlay one another. The edges of the first section 32 and the second section 34 are provided with respective parts of a hook-and-eye connection mechanism 38. Notably, the side edges are not provided with a connection mechanism.

The first surface is provided with two tube retaining devices 40, in the form of clips 42 that are made from resiliently yieldable plastics material to allow for the medical tubes to be ‘snap-fitted’ into the clips 42 and releasably retained therein.

FIGS. 5 and 6 show a second embodiment of the present invention in the form of a device 10′ having many of the same features as the device of FIGS. 1 to 3. However, this second embodiment comprises a back fastening vest 12′ having a zipper 60 to enable the device to be readily put on and removed. Whilst the zipper 60 is shown at the rear of the device, it may be positioned on the side in some arrangements. Additionally, in the embodiment of these figures, the connection mechanism between the straps 18′ and the front panel 14′ comprises a click fastener 19.

The zipper may be orientated in different directions, for example, vertically on the vest, or diagonally, and it may be positioned on the front panel in some arrangements.

FIG. 7 shows a third embodiment of the present invention, wherein the vest is shown in use. A medical tube 70 passes through the aperture 28″ and into the front pocket 30″. In this embodiment, the straps 18″ are fixed to the front panel 14″, however, the apertures 28″ are sufficiently large to allow the medical tube to be positioned at a variety of heights, relative to the front panel 14″ of the vest.

The vest may comprise a loop or slot in the front panel through which the medical tube may pass to provide further support.

Where the vest is made from two layers of material, the retaining means may be mounted upon one layer and pass through the second layer to protrude into the pocket. Padding may be positioned second surface or, where the second surface comprises two layers, it may be positioned between the two layers and extend through an aperture in one of those layers. Where two layers are employed, it may be advantageous to position cushioning material, such as foam, between the tube retaining device and the layer that contacts the user's skin, in use.

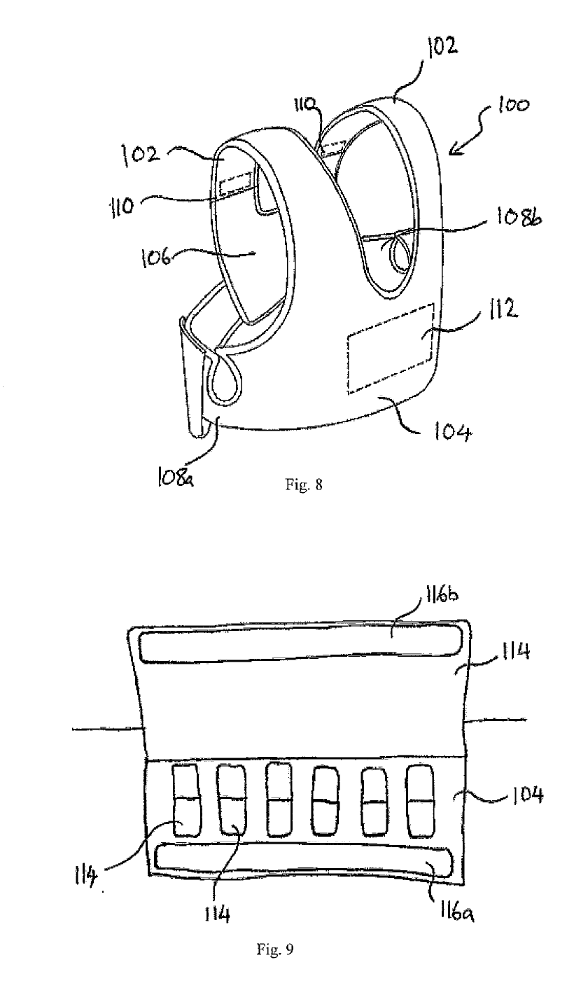

FIGS. 8 and 9 show a device 100 comprising shoulder straps 102 that connect a front panel 104 with a back panel 106. Lateral straps 108a and 108b are provided about the sides of the device 100 to connect the front panel 104 with the back panel 106 at a connection section (not shown) on the back panel 106, but that may be in the form of loops through which the lateral straps 108 can pass and/or hook-and-eye fasteners. The shoulder straps 102 comprise releasable openings 110 with corresponding hook-and-eye sections that allow the shoulder straps 102 to be readily released and connected to allow a user to easily put on the device 100 and/or to adjust the length of the shoulder straps 102. An internal pocket 112 is provided on the inside of the front panel 104.

The internal pocket 112 comprises a flap 114 that can be readily opened to expose the inside of the pocket 112. FIG. 9 shows the internal pocket 112 in an open position with flap 114 raised. Within the pocket 112 are a series of loops 114 in which the end of a medical tube can be held according to the length of the tube and its position on the patient. Hook-and-eye fastenings 116a and 116b are provided to releasably retain the pocket 112 in a closed position.

The top surface of the pocket may be larger than the lower surface to enable a user to readily grip the pocket in order to open it.

1. A medical tube holder comprising a vest and at least one sealable pocket located on the vest, wherein the inside of the pocket is provided with at least one tube retaining device into which the end of a medical tube may be releasably retained.

2. A medical tube holder according to claim 1, wherein the tube retaining device is a clip into which a medical tube can be received.

3. A medical tube holder according to claim 1, wherein the vest comprises at least one connection system to allow the vest to be opened.

4. A medical tube holder according to claim 3, wherein the vest is provided with at least one sealable opening.

5. A medical tube holder according to claim 4, wherein the at least one sealable opening comprise one or more of: a zipper; hook-and-eye fastener; and
snap-fit connectors.

6. A medical tube holder according to claim 1, wherein the vest comprises at least one shoulder strap that is adjustable in length.

7. A medical tube holder according to claim 1, wherein the vest comprises at least one aperture through which a medical tube may pass.

8. A medical tube holder according to claim 7, wherein the area of the at least one aperture is adjustable.

9. A medical tube holder according to claim 1, wherein the vest comprises a plurality of pockets in different positions.

10. A medical tube holder according to claim 1, wherein the pocket comprises;
a first surface; and
a second surface designed to at least partially overlay the first surface;
wherein the first surface and the second surface are pivotally connected along at least a first edge, and
wherein at least a second edge of the first surface and/or second surface is provided with connecting means for releasably connecting the surfaces together.

11. A medical tube holder according to claim 1, wherein the vest comprises a front panel and a back panel with two shoulder straps connecting an upper part of the front panel to an upper part of the back panel and a head aperture between the shoulder straps.

12. A medical tube holder according to claim 1, wherein padding is provided adjacent the pocket.
